(19) United States
(12) Patent Application Publication
(10) Pub. No.: US 2011/0256080 A1
(43) Pub. Date: Oct. 20, 2011

(54) Easily appliable, storage stable, radiation-curable, pigmented, artificial nail gel coatings

(75) Inventors: Joseph E. Kozachek, Newtown Square, PA (US); George M. Lein, Elkton, MD (US)

(73) Assignee: MYCONE DENTAL CORPORATION

(21) Appl. No.: 12/804,253

(22) Filed: Jul. 16, 2010

Related U.S. Application Data
(63) Continuation-in-part of application No. 12/798,953, filed on Apr. 14, 2010.

(51) Int. Cl. A61K 8/81; A61Q 3/02

(52) U.S. Cl. 424/61

(57) ABSTRACT

Limited shelf life due to pigment settling and the formation of hard packs in pigmented UV curable nail gels is solved by the present invention which describes a composition, method, and use of a UV curable, thixotropic, radiation curable, low viscosity gel comprised of a formulation containing thixotropic additive(s) and in some cases dispersants for additional dispersion stability. The thixotropic pigmented gel has prolonged shelf life and long time storage at ambient conditions. It also contains negligible amounts of unreactive solvent. The thixotropic additive also changes the rheological properties of the gel, which allows the nail gel to be easily applied to nails at a lower viscosity due to shear thinning.

This patent application is a continuation-in-part of application Ser. No. 12/798,953, filed Apr. 14, 2010.

### BACKGROUND OF THE INVENTION

This invention relates to the field of radiation-curable gels useful for cosmetic adornment of natural nails, artificial fingernails, toenails and artificial nail extensions.

The use of radiation-curable gels in formation of nail enhancements or artificial nails has been an important part of the cosmetic industry since it was first introduced. U.S. Pat. No. 4,682,612, describing the use of actinic radiation-curable compositions suitable for preparation of artificial nails, is representative of this technology.

Ultra-violet radiation (UV) is the most conventional form of radiation used to cure gels in this art, however, visible light curing systems are also known. Professional nail technicians most typically apply UV curable gels designed for sculpting nails. Such UV-curable gels are usually composed of acrylic or methacrylic monomers and oligomers in a gel-like state that requires curing under a UV lamp. Such nail finishes can be applied directly to natural fingernails or toenails, or alternatively can be applied to nail extensions bonded to fingernails. In many cases, the artificial nails are coated with conventional nail polish after they are cured.

In order to avoid the need to coat the artificial nails or natural nails with conventional nail polish containing high levels of solvent, in more recent years, the preparation of gels containing colorants, particularly pigments, has become known in this art.

Present color pigmented gel formulations are highly viscous and are not as easy to apply as per the ease of applying nail polish for example. Lower viscosity gels are preferred since their application properties are similar to standard nail polishes. However, the use of lower viscosity gels, leads to colored gel formulations having a limited shelf life due to pigment settling and the formation of hard packs. Once the hard pack is formed, it is difficult or nearly impossible to resuspend the pigment into the gel as a homogeneous composition.

Sculpting, or builder gels, are very difficult to apply due to their high viscosity and they often need to be filed down and reapplied multiple times to build up a nail. Low viscosity UV curable gels, as per the composition of the invention stated herein, can be applied as a thin coat and are typically applied as a single coating. A second coating is optional. Therefore, there is an unmet need in the art for a low viscosity UV curable nail gel coating with good control of flow when applied to nails in combination with a homogeneous distribution of color pigment that does not easily form hard packs upon storage. Storage shelf life at ambient conditions is critical for a sellable product.

Prior references demonstrate the addition of agents to modify the rheological properties of solvent based nail polishes and high viscosity UV sculpting gels, but say nothing in regards to an agent that will impart a reduction in high shear viscosity and impart stability to pigmented low viscosity UV curable gel coatings. U.S. Pat. No. 6,244,274B1 “Thixotropic Polymerizable Nail Sculpting Compositions” (Sirdesai et. al.) claims a single composition and method to modify the rheological properties of a reactive UV curable sculpting gel. While rheological modification of a builder gel may make it easier to apply, the viscosity at application of this type of gel is necessarily much higher than that of a coating gel in order to allow for the building of the artificial nail. The invention in U.S. Pat. No. 6,244,274B1 discloses a sculpting high viscosity UV curable gel containing low or no pigments. The levels exemplified in “274B1” are typical of those used to provide a natural look. In addition, color pigments are not exemplified and there is no mention of pigment stability upon storage.

Current UV curable gels containing pigments are of high viscosity making them difficult to apply and the current state of the art UV curable gels of lower viscosity have been either without pigment or contain only very low levels of pigment.

Although, U.S. Pat. No. 6,555,096, “Nail Enamel Composition Containing a Urea-modified Thixotropic Agent in a Solvent System” (Carrion et. al) claims a nail enamel and a urea modified thixotropic agent in a solvent and mentions in the description that the thixotropic agent acts to suspend the colorant, the “096” patented formulation is not a UV curable gel. U.S. Pat. No. 4,222,908, “Thixotropic Nail Enamel” by Ikeda et. al. also utilizes a gelling agent (montmorillonite clay) in a thixotropic nail enamel. Furthermore, Ikeda describes the use of such gelling agents as a way to prevent separation of pigments and pearl essences.

It should also be stated here that large quantities of organic solvents, which are undesirable in UV curable gels, are used in the relatively low viscosity nail enamels. For example in U.S. Pat. No. 5,985,951, Cook states in the detailed description of his invention which happens to be a UV curable coating, column 8, line 10, the invention needs about 65-75 wt % suitable solvent to solubilize the “solvent based modified cellulose ester coating composition”. This is an example of typical amounts of unreactive solvent in commercial nail coatings. The solvent, in “951”, in addition to the coating, is applied to nails and emits significant amounts of unpleasant and potentially harmful solvent vapors. In comparison, in the invention herein, we anticipate levels of unreactive solvent<8 wt %, and preferably <5 wt % and most preferably <1 wt % remaining in the final UV nail gel. This attribute allows the use of our product to be preferred in Nail Salons and anywhere where the gel is applied in close spaces such as in malls and small neighborhood stores.

In U.S. Pat. No. 6,051,242, Patel describes a quick drying nail polish coating composition, containing a thixotropic additive, which is comprised of two different solvent dissolved polymer systems for primary and secondary film formation. The nail polish composition is comprised of solvent levels from 8 to 80 WI percent and it is stated in the specification that it is most preferred to contain 45-55% solvent. The nail polish material also only contains about 1% to 20% reactive species (the reactive species is selected from a specific group of monomers, oligomers, and polymers) compatible with the primary and secondary film forming polymers. The Patel composition is a typical low viscosity nail polish (with a viscosity of no more than 4.8 Poise at low shear rates, per the specification) and it is not UV cured. UV cured nail gels, even representative low viscosity UV gels, as per the invention stated herein, typically have viscosities of >10 Poise at low shear rates. The Patel composition also contains a thixotropic agent, but the purpose of the thixotropic agent is just to gel the base composition or lacquer.

In addition, a considerable advantage of the use of the UV nail gel for the customer and the person performing the application is the reduced time needed to harden. A customer can spend up to an hour waiting for the solvent in nail enamel to evaporate, while the gel is set in 3 minutes or less.

### SUMMARY OF THE INVENTION

The problems of limited shelf life due to pigment settling and the formation of hard packs in low viscosity, low VOC, UV curable gels are solved by the present invention which comprises in one aspect a composition comprised of a low viscosity radiation curable color gel coating comprised of a formulation containing the addition of thixotropic additive(s) and in some cases dispersants for additional dispersion stability. The thixotropic color pigmented UV curable gel has prolonged shelf life and long time storage at ambient conditions. In another embodiment, dispersants can be added to improve both the shelf life and to allow for resuspension of pigment to further improve shelf life. In addition, this invention resolves an additional long time problem with its ease of application as it behaves similarly to a commercial nail polish in terms of application to nails but without significant amounts of unpleasant and potentially harmful solvent vapors. Sculpting or builder gels need to be applied by a highly trained professional; the invention herein can be applied by a novice.

In yet another aspect, the invention claims the use of such a highly shelf stable colored UV-curable artificial nail gel.

### DETAILED DESCRIPTION OF PREFERRED EMBODIMENTS

According to the invention herein, shelf stable, pigment containing, low viscosity UV curable nail gel coatings can be successfully prepared by utilizing thixotropic additive(s) to prepare such gels. In another embodiment, dispersants can be added to improve both the shelf life and to allow for resuspension of pigment to further improve shelf life at ambient conditions. The thixotropic additive builds in a reduction of high shear viscosity allowing the application of a uniform coating without adversely affecting the rheological properties of the gel. This allows the nail gel to be easily applied to nails at a lower viscosity due to shear thinning properties.

For good shelf stability at ambient conditions (about room temperature or around 25 degrees Celsius) the viscosity of the system at rest must be sufficient to keep pigments suspended as defined by Stoke's law below.

$${V}_{s} = \frac{2}{9} \frac{( {ρ}_{p} - {ρ}_{f} )}{μ} {gR}^{2}$$

where:
Vₛ is the particles' settling velocity (m/s) (vertically downwards if ρₚ>ρ_{f}, upwards if ρₚ<ρ_{f}),
g is the gravitational acceleration (m/s²),
ρₚ is the mass density of the particles (kg/m³), and
ρ_{f} is the mass density of the fluid (kg/m³).
μ is the fluid's viscosity (in [kg m⁻¹ s⁻¹]),
R is the radius of the spherical object (in m), and
   Shear rates under these “system at rest” very low shear conditions are in the 10⁻⁴/sec to 10⁻⁶/sec range (as stated in the Rheology Modifiers handbook, David & Braun and Meyer R. Rosen, William Andrew Publishing, 1999, Pg 17): Thus, the thixotropic additive(s) must impart sufficient viscosity under low shear rate conditions to prevent pigment settling and show viscosity reduction upon the applied shear such that good application properties are obtained. Ease of application requires a viscosity under a shear rate of 70/sec of less than 40 poise with less than 30 poise being preferred and less than 20 poise being most preferred. The shear rate for application is generally in the range of 50/sec-1000/sec.

The UV-curable artificial nail gels can be comprised of a wide variety of compounds containing one or more radical polymerizable unsaturated double bonds. Typical examples include esters and amides of acrylic and methacrylic acid. The esters of acrylic and methacrylic acid are herein termed (meth)acrylic ester. Specific but not limiting examples of mono methyl(meth)acrylic esters include: methyl(meth)acrylate, ethyl(meth)acrylate hydroxypropyl(meth)acrylate, ethyl(meth)acrylate, butyl (meth)acrylate, hydroxy ethyl(meth)acrylate, butoxyethyl(meth)acrylate, diethylaminoethyl(meth)acrylate, 2-ethylhexyl(meth)acrylate, ethoxyethyl (meth)acrylate, t-butyl aminoethyl(meth)acrylate, methoxyethylene glycol (meth)acrylate, phosphoethyl(meth)acrylate, methoxy propyl(meth)acrylate, methoxy polyethylene glycol(meth)acrylate, phenoxyethylene glycol (meth)acrylate, phenoxypolyethylene glycol (meth)acrylate, 2-hydroxy-3-phenoxypropyl(meth)acrylate, 2-(meth)acryloxyethylsuccinic acid, 2-(meth)acryloylethylphthalic acid, 2-(meth)acryloyloxypropylphthalic acid, stearyl(meth)acrylate, isobornyl(meth)acrylate, 3-chloro-2-hydroxypropyl(meth)acrylate, tetrahydrofurfuryl(meth)acrylate, (meth)acrylamides and allyl monomers. Specific but not limiting examples of Bifunctional methacryl esters include: 1,4-butanediol di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octane diol di(meth)acrylate, glycerol di(meth)acrylate, ethylene glycol di(meth)acrylate, triethylene glycol di(meth)acrylate, polyethylene glycol di(meth)acrylate, polypropylene glycol di(meth)acrylate, ethoxylated propylene glycol di(meth)acrylate, ethoxylated polypropylene glycol di(meth)acrylate, polyethoxypropoxy di(meth)acrylate, ethoxylated bisphenol A di(meth)acrylate, propoxylated bisphenol A di(meth)acrylate, propoxylated ethoxylated bisphenol A di(meth)acrylate, bisphenol-A glycidyl methacrylate, tricyclodecanedimethanol di(meth)acrylates glycerin di(meth)acrylate, ethoxylated glycerin di(meth)acrylate, bis acrylamides, bis allyl ethers and allyl(meth)acrylates. Examples of tri and or higher (meth)acryloyl esters include trimethylolpropane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol penta(meth)acrylate, dipentaerythritol hexa(meth)acrylate, and ethoxlated iscyanuric acid tri(meth)acrylates.

Urethane(meth)acrylates, useful in the present invention, have at least two or more acryl or methacryl groups and a urethane group. Examples include: urethanes based on aliphatic, aromatic, polyester, and polyether polyols and aliphatic, aromatic, polyester, and polyether diisocyanates capped with (meth)acrylate end-groups. Isocyanate prepolymers can also be used in place of the polyol-diisocyanate core. Epoxy(meth)acrylates and epoxy urethane (meth)acrylates, useful in the present invention, have at least two or more acryl or methacryl groups and, optionally, a urethane group. Examples include epoxy(meth)acrylates based on aliphatic or aromatic epoxy prepolymers capped with (meth)acrylate end-groups. A aliphatic or aromatic urethane spacer can be optionally inserted between the epoxy and the (meth)acrylate endgroup(s). Acrylated polyester oligomers, useful in the present invention, have at least two or more acryl or methacryl groups and a polyester core. Acrylated polyether oligomers, useful in the present invention, have at least two or more acryl or methacryl groups and a polyether core. Acrylated acrylate oligomers, useful in the present invention, have at least two or more acryl or methacryl groups and a polyacrylic core. These reactive urethanes, epoxies, polyesters, polyethers and acrylics are available from several suppliers including BASF Corporation, Bayer MaterialScience, Bomar Specialties Co, Cognis Corporation, Cytec Industries Inc, DSM NeoResins, Eternal Chemical Co, Ltd, IGM Resins, Rahn A G, Sartomer USA, LLC, and SI Group, Inc.

In addition to the above-described (meth)acrylate-based polymerizable monomers, other polymerizable monomers, oligomers or polymers of monomers which contain at least one free radical polymerizable group in the molecule may be used without any limitations in the curable gel. These monomers may contain other groups such as carboxyl groups to improve adhesion.

A compound having at least one free radical polymerizable group includes not only a single component but also a mixture of polymerizable monomers. Thus, combinations of two or more materials containing free radical polymerizable groups may be used in combination.

The gels also contain a photoinitiator. Examples of these include: benzyl ketones, monomeric hydroxyl ketones, polymeric hydroxyl ketones, alpha-amino ketones, acyl phosphine oxides, metallocenes, benzophenone, benzophenone derivatives, and the like. Specific examples include: 1-hydroxy-cyclohexylphenylketone, benzophenone, 2-benzyl-2-(dimethylamino)-1-(4-(4-morpholinyl)phenyl)-1-butanone, 2-methyl-1-(4-methylthio)phenyl-2-(4-morpholinyl)-1-propanone, diphenyl-(2,4,6-phosphine oxide, phenyl bis(2,4,6-trimethylbenzoyl) phosphine oxide, benzyl-dimethylketal, isopropylthioxanthone, and mixtures thereof.

Photo accelerators such as aliphatic or aromatic amines may also be included in the gel as well as fillers, inhibitors, plasticizers, polymers, and adhesion promoters.

Suitable pigments which can be incorporated into the color concentrates include barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides, Preferred pigments include D&C Black No. 2, D&C Black No. 3, FD&C Blue No. 1, D&C Blue No. 4, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30. D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, as well as others listed on the FDA color additives website, and Annex IV of the Cosmetic Directive 76/768/EEC, Coloring Agents Permitted in Cosmetics as of Mar. 1, 2010.

Pigment levels in the composition can be from greater than 0.1 wt % up to as much as 20-wt %. Colored pigments are preferred from 0.5 up to 10-wt %. Mixtures of TiO2 and colored pigments are most preferred.

A thixotropic additive is defined herein as an additive that when mixed with a relatively low viscosity gel imparts shelf stability to the pigmented gel. The pigment does not readily fall out of the gel to form a hard pack. A hard pack is essentially the material (mainly pigment) that settles and can not be easily redispersed by low shear methods such as shaking or rolling. In most cases the hard pack (pigments) cannot be re-incorporated back into the nail gel. These dry pigment agglomerates, if they are resuspended into the gels and applied with the coating onto the nails, tend to form surface defects in the nail gel coating and poor appearance. In the invention herein, when particles do settle over time in the gel, they do not form a hardpack and they can be easily resuspended with gentle mixing such as shaking and rolling. The thixotropic additive preferably imparts shear thinning properties such that a viscosity reduction of at least a factor of 1.5 occurs over the range of shear from that when the sample is at rest to that encountered under use conditions, e.g., 10⁻⁶/sec to 70/sec, i.e.—the gel is thick (viscous) under normal storage conditions, but flows (becomes thin, less viscous) when stressed such as applying the gel to nails: The thixotropic additive changes the rheological properties of the gel. The thixotropic additive also imparts pseudoplasticity to the system. A thixotropic fluid displays a decrease in viscosity over time at a constant shear rate, while a pseudoplastic fluid displays decreasing viscosity with increasing shear rate. We mention the thixotropic nature of the nail gels here in this application, but one can also state that the nail gels with the thixotropic additive are exhibiting non-Newtonian pseudoplastic behavior.

Thixotropic additives useful in this invention include inorganic and organic based materials. Compositions containing inorganic based thixotropic additives demonstrate higher yield stress relative to viscosity obtained at applied shear rates compared to compositions containing organic thixotropic additives. In practical terms, this is typified by a steeper reduction in viscosity upon adding shear to the system. This allows good fluidity and handling even at low shear rates such as 2/sec. Examples of inorganic based materials useful in the invention include but are not limited to: calcium, zinc or aluminum stearate, silica, fumed silica such as that available as Aerosil® from Evonik Industries or Cab-O-Sil® available from Cabot Corporation, diatomaceous earth, bentonite clay, kaolinite, pyrophyllite, sericite, saponite, smectic/vermiculites (montmorillinite, beidillite, nontronite, hectorite and saponite), organic modified clays including but not limited to: stearalkonium or disteardialkonium bentonite and hectorite and others that are available from Elementis Specialties under the trade name of Bentone® and Garamites® from Rockwood Additives such as Garamite® 1958, talc, mica, zirconium oxide, zinc oxide and magnesium oxide. Examples of organic based thixotropic additives include but are not limited to: hydrogenated castor oils, hydrogenated castor oil waxes, inorganically modified castor oils, organically modified castor oils such as those sold by Elementis Specialties under the Thixcin® trademark, triglycerides such as glyceryl tri-12-hydroxy stearate, polyamides and modified polyamides such as 12-hydroxystearic acid diamide of ethylene diamine, 12-hydroxystearicacid diglycolamide, N-stearyl ricinoleamide, N-stearyl stearamide and other polyamide waxes. Included in these polyamide materials are those sold commercially by Kusumoto Chemicals Industries under the Disparlon® trademark, by Lehmann and Voss under the Luvotix® trademark, by Elementis Specialties under the Thixatrol® trademark, polyethylene oxide waxes, urea urethanes believed to be exemplified by those sold by Byk Incorporated as, for example, by Byk-410, Byk-411, and Byk-420, acrylic resins, amine salts of polymeric polyesters, salts of linear polyaminoamide and polymeric polyester, amide solutions of polycarboxylic acid, alkyl sulfonate, alkylallyl sulfonate, colloidal ester, polyester resin such as those sold by Elementis Specialties under the Thixatrol® trademark, phenol resin, melamine resin, epoxy resin, urethane resin, styrene butadiene polymers, polyimide resin, and polyester amides. Materials such as those sold by Byk under the trademarks of Anti-Terra® and Bykumen® can also be used.

Thixotropic additives can be used at amounts from 0.1 to 10 wt. %. It is preferred to use quantities from 0.5 to 5.0 wt. % and more preferred to use amounts of 0.5 to 3.0 wt %. Although organic thixotropic additives are effective, the preferred thixotropic additives are organic modified clays and organic modified or unmodified silicas. As stated above, the inorganics provide a steeper reduction in viscosity upon added shear. Mixtures of inorganic based thixotropic additives and organic based thixotropic additives can also be used.

A dispersant additive is defined herein as an additive that is either a non-surface active polymer or a surface-active substance added to a gel suspension to improve the separation of particles and to prevent agglomeration. Dispersants consist normally of one or more surfactants or polymers.

Examples of suitable dispersants include but are not limited to: dispersants sold by Buckman Laboratories under the Busperse® trademark, dispersants sold by Byk under the Disperbyk® trademark, dispersants sold by Lubrizol under the Solsperse® trademark, dispersants sold by BASF under the EFKA® trademark, dispersants sold by Kyoeisha Chemcial Co. under the Flowlen® trademark, dipersants sold by Ajinomoto under the Ajisper® trademark, dispersants sold by Cognis under the Texaphor® trademark, dispersants sold by Cytec Industries under the Aerosol® trademark, dispersants sold by Ethox chemicals, dispersants sold by San Nopko, dispersants sold by Kusumoto Chemical under the Disparlon® trademark and dipersants sold by Evonik under the Tego® trademark.

By the term “gel,” we mean a radiation-curable composition comprising photoinitiator, ethylenically unsaturated monomers and/or oligomers, having a viscosity suitable for coating natural or artificial nails, or artificial nails and extensions, as well as adorning such nails.

Unreactive solvent is defined herein as a volatile species (such as a low boiling temperature liquid) that evaporates from typical nail enamel formulations and in final UV cured formulations and serves no reactive function in the formulation per se. In contrast, examples of reactive solvent are reactive monomers and/or other reactive species remaining in the formulation. The formulations described herein are comprised of predominantly reactive monomers and or oligomers and mixtures thereof, and these are utilized in amounts of >60 wt % in the formulation, which upon UV activation are the primary components of the resulting UV cured nail gel. The reactive monomers and or oligomers can act as “reactive solvents” as they can solubilize different components in the composition.

There are many possible embodiments of the nail gel. In some embodiments the gel is comprised of 50-70% by weight of an aliphatic polyester based urethane multimethacrylate oligomer, 15-25% by weight 2-hydroxyethyl methacrylate (HEMA), 15-25% by weight 2-hydroxypropyl methacrylate (HPMA), 1-5% (photoinitiator) and 0.1-10% by weight of organically modified clay (thixotropic additive), and 0.1-10% by weight FD&C Red #7 Calcium Lake pigment. In certain other embodiments the thixotropic agent is 0.1-10% by weight silica. Other embodiments can be comprised of aliphatic polyester based urethane diacrylate oligomer and FD&C Red #6 lake pigment.

In the below examples, which further define the embodiments, the gel viscosities are measured at 25° C., 2/sec shear and 70/sec shear, on a TA Instruments AR500 Rheometer. 2/sec represents a low shear rate and 70/sec represents a high shear rate. In terms of stability of the UV gel, we define syneresis as separation of the pigment and the gel to leave a clear layer at the top of the gel. Ultimately, in some cases a compacted solid hard pack is formed.

### EXAMPLES

### Example 1

To 125 g of a UV-curable gel comprising 58% by weight of an aliphatic polyester based urethane multimethacrylate oligomer, 20% by weight hydroxyethylmethacrylate, 20% by weight hydroxypropyl methacrylate and 2% photoinititator was added 25 g of a dispersion which had been prepared by shearing at 10000 rpm (using a Biohomogenizer available from Biospec Products) 14 grams of Aerosil® 300 (available from Evonik Degussa GmbH) into 686 g of the same UV curable gel described above. To 44.7 g of the resulting dispersion was added 5.25 g of a 24% wt/wt dispersion of FD&C Red #7 Calcium Lake in a diacrylate monomer. The resulting composition had a viscosity at 25° C. of 18 poise at 2/sec shear rate, and 13 poise at 70/sec shear rate. The mixture was allowed to stand at ambient temperature and periodically checked for settling. After 26 days, no settling had occurred.

### Comparative Example 1

To 44.7 g of the UV curable gel used in Example 1 (to which no silica dispersion had been added) was added 5.25 g of the Red #7 Calcium Lake dispersion used in Example 1. After standing 5 days, a clear pigment free layer had formed on the top of the sample.

### Example 2

To 125 g of a UV-curable gel comprising 58% by weight of an aliphatic polyester based urethane multimethacrylate oligomer, 20% by weight hydroxyethylmethacrylate, 20% by weight hydroxypropyl methacrylate and 2% photoinititator was added 25 g of a dispersion containing the thixotropic additive (Aerosil® 300). The dispersion had been prepared by shearing at 10000 rpm (using a Biohomogenizer available from Biospec Products) 14 grams of Aerosil® 300 (available from Evonik Degussa GmbH) into 686 g of the same UV curable gel described above. To 47.7 g of the resulting dispersion was added 2.3 grams of a 58% wt/wt dispersion of titanium dioxide in a diacrylate monomer. The resulting composition had a viscosity at 25° C. of 20 poise at 2/sec shear rate and 16 poise at 70/sec shear rate. The mixture was allowed to stand at ambient temperature and periodically checked for settling. After 26 days, no settling had occurred.

### Comparative Example 2

To 47.7 g of the UV curable gel used in Example 2, to which no silica dispersion had been added, was added 2.3 g of the titanium dioxide dispersion used in Example 1. After standing 5 days, nearly all pigment had settled.

### Example 3

To 122 g of a UV-curable gel comprising 58% by weight an aliphatic polyester based urethane multimethacrylate oligomer, 20% by weight hydroxyethylmethacrylate, 20% by weight hydroxypropyl methacrylate and 2% photoinititator was added 28 g of a dispersion which had been prepared by shearing: 32 g of Garamite® 1958, available from Rockwood Specialties, and 368 g of the same UV curable gel as described above in a Cowles laboratory mixer at 1500 rpm. To 44.7 g of the resulting dispersion was added 5.3 g of a 24% wt/wt dispersion of FD&C Red #7 Calcium Lake Pigment dispersed in a diacrylate monomer. The resulting composition had a viscosity at 25° C. of 21 poise at 2/sec shear rate and 16 poise at 70/sec shear rate. The mixture was allowed to stand at ambient temperature and periodically checked for settling. After 26 days, no settling had occurred.

### Example 4

To 450 g of a UV-curable gel comprised of 58% by weight of an aliphatic polyester based urethane multimethacrylate oligomer, 20% by weight hydroxyethylmethacrylate, 20% by weight hydroxypropyl methacrylate and 2% photoinitiator was added 150 g of a dispersion which was prepared by shearing 40 g of a bentonite clay Garamite 1958 available from Rockwood Specialties, and 460 g of the same UV curable gel as described above in a Cowles laboratory mixer at 1500 rpms. To 45.2 g of the resulting dispersion was added 4.8 g of Red #7 LT Paste concentrate available from Tevco, Inc, which is Red #7 Lake Pigment dispersed in a mixture of ethyl acetate, butyl acetate, isopropyl alcohol, nitrocellulose and Red 7 lake. The resulting composition had a viscosity at 25° C. of 11.8 poise at 2/sec shear rate and 7.9 poise at 70/sec shear rate. The mixture was subdivided and one sample was maintained in a 50° C. oven and the other at ambient temperature and periodically checked for settling. After 34 days, neither sample showed signs of settling.

### Example 5

To 406.2 g of a UV-curable gel comprised of 58% by weight of an aliphatic polyester based urethane multimethacrylate oligomer, 20% by weight hydroxyethylmethacrylate, 20% by weight hydroxypropyl methacrylate and 2% photoinitiator was added 94 g of a dispersion which was prepared by shearing 40 g of a bentonite day Garamite 1958 available from Rockwood Specialties, and 460 g of the same UV curable gel as described above in a Cowles laboratory mixer at 1500 rpms. To 95.2 g of the resulting dispersion was added 6.8% of a color mixture consisting of 0.39% Yellow #5 Paste, 0.8% Red 34 Paste and 5.7% Red #7 LT Paste all available from Tevco, Inc. All pastes contain the lake form of the pigment, dispersed in a mixture of ethyl acetate, butyl acetate, isopropyl alcohol and nitrocellulose. The resulting mixture had a viscosity at 25° C. of 14 poise at a shear rate of 2/see shear rate and 10 poise at 70/sec shear rate. The mixture was subdivided and one sample was maintained in a 50° C. oven and the other at ambient temperature with periodic checks made for settling. After 33 days, neither sample showed signs of settling.

The present invention, therefore, is well adapted to carry out the objects and attain the ends and advantages mentioned, as well as others inherent therein. While the invention has been depicted and described and is defined by reference to particular preferred embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described preferred embodiments of the invention are exemplary only and are not exhaustive of the scope of the invention. Consequently, the invention is intended to be limited only by the spirit and scope of the appended claims, giving full cognizance to equivalents in all respects.

What is claimed is:

1. A shelf stable, pigment containing, UV-curable, low viscosity nail gel coating composition useful for adornment of natural and artificial nails and artificial nail extensions comprising: a pigment or mixture of pigments, reactive monomers selected from one or more ethylenically unsaturated monomers, and one or more ethylenically unsaturated oligomers, or mixtures thereof, a photoinitiator, and one or more thixotropic additives, with the gel having a high shear viscosity less than 70 poise.

2. A shelf stable, pigment containing, UV-curable, low viscosity nail gel coating composition useful for adornment of natural and artificial nails and artificial nail extensions comprising: a pigment or mixture of pigments, reactive monomers selected from one or more ethylenically unsaturated monomers, and one or more ethylenically unsaturated oligomers, or mixtures thereof, a photoinitiator, and one or more thixotropic additives, with the gel having a high shear viscosity less than 70 poise and a viscosity reduction of at least a factor of 1.5 over the range of shear from when the sample is at rest to that encountered under use conditions.

3. The composition of claim 1 that is shelf stable and pigment does not settle out wherein the high shear viscosity is less than 50 poise.

4. The composition of claim 1 that is shelf stable and pigment does not settle out wherein the high shear viscosity is less than 30 poise.

5. The composition of claim 1 that is shelf stable and pigment does not settle out wherein the high shear viscosity is less than 20 poise.

6. The composition of claim 1 that is shelf stable and pigment does not settle out wherein the high shear viscosity is less than 70 poise and the low shear viscosity is greater than 15 poise.

7. The composition according to claim 1 that is shelf stable and pigment does not settle out at a high shear viscosity less than 50 poise and a low shear viscosity greater than 20 poise.

8. The composition according to claim 1 that is shelf stable and pigment does not settle out at a high shear viscosity less than 50 poise and a low shear viscosity greater than 30 poise.

9. The composition according to claim 1 that is shelf stable and pigment does not settle out at low shear rates from 10⁻⁴/sec to 10⁻⁶/sec.

10. The composition according to claim 1 where the reactive monomers selected from one or more ethylenically unsaturated monomers, and one or more ethylenically unsaturated oligomers, or mixtures thereof, is in amounts greater than 60 wt %.

11. The composition according to claim 1 where the reactive monomers selected from one or more ethylenically unsaturated monomers, and one or more ethylenically unsaturated oligomers, or mixtures thereof, is in amounts greater than 60 wt %, and contains less than 8 wt % unreactive solvent.

12. The composition according to claim 1 where the reactive monomers selected from one or more ethylenically unsaturated monomers, and one or more ethylenically unsaturated oligomers, or mixtures thereof, is in amounts greater than 60 wt %, and contains less than 4 wt % unreactive solvent.

13. The composition according to claim 1 where the reactive monomers selected from one or more ethylenically unsaturated monomers, and one or more ethylenically unsaturated oligomers, or mixtures thereof, is in amounts greater than 60 wt %, and contains less than 1 wt % unreactive solvent.

14. The composition according to claim 1 where the reactive monomers selected from one or more ethylenically unsaturated monomers, and one or more ethylenically unsaturated oligomers, or mixtures thereof, is in amounts greater than 80 wt %.

15. The composition according to claim 1 where the reactive monomers selected from one or more ethylenically unsaturated monomers, and one or more ethylenically unsaturated oligomers, or mixtures thereof, is in amounts greater than 80 wt %, and contains less than 8 wt % unreactive solvent.

16. The composition according to claim 1 where the reactive monomers selected from one or more ethylenically unsaturated monomers, and one or more ethylenically unsaturated oligomers, or mixtures thereof, is in amounts greater than 80 wt %, and contains less than 4 wt % unreactive solvent.

17. The composition according to claim 1 where the reactive monomers selected from one or more ethylenically unsaturated monomers, and one or more ethylenically unsaturated oligomers, or mixtures thereof, is in amounts greater than 80 wt %, and contains less than 1 wt % unreactive solvent.

18. The composition according to claim 1 where the reactive monomers selected from one or more ethylenically unsaturated monomers, and one or more ethylenically unsaturated oligomers, or mixtures thereof, is in amounts greater than 90 wt %.

19. The composition according to claim 1 where the reactive monomers selected from one or more ethylenically unsaturated monomers, and one or more ethylenically unsaturated oligomers, or mixtures thereof, is in amounts greater than 90 wt %, and contains less than 8 wt % unreactive solvent.

20. The composition according to claim 1 where the reactive monomers selected from one or more ethylenically unsaturated monomers, and one or more ethylenically unsaturated oligomers, or mixtures thereof, is in amounts greater than 90 wt %, and contains less than 4 wt % unreactive solvent.

21. The composition according to claim 1 where the reactive monomers selected from one or more ethylenically unsaturated monomers, and one or more ethylenically unsaturated oligomers, or mixtures thereof, is in amounts greater than 90 wt %, and contains less than 1 wt unreactive solvent.

22. The composition of claim 1 containing less than 8 wt % unreactive solvent.

23. The composition of claim 1 containing less than 4 wt % unreactive solvent.

24. The composition of claim 1 containing less than 1 wt % unreactive solvent.

25. The composition according to claim 1 wherein the thixotropic additive can be from the group consisting of: calcium, zinc or aluminum stearate, silica, fumed silica, diatomaceous earth, bentonite clay, kaolinite, pyrophyllite, sericite, saponite, smectic/vermiculites (montmorillinite, beidillite, nontronite, hectorite and saponite), organic modified bentonite and hectorite such as stearyl alkonium hectorite, disteardialkonium hectorite, talc, mica, zirconium oxide, zinc oxide, magnesium oxide, organic modified clays, and Garamites® such as Garamite® 1958.

26. The composition according to claim 1 wherein the thixotropic additive can be from the group consisting of hydrogenated castor oils, hydrogenated castor oil waxes, inorganically modified castor oils, organically modified castor oils, triglycerides such as glyceryl tri-12-hydroxy stearate, polyamides and modified polyamides such as 12-hydroxystearic acid diamide of ethylene diamine, 12-hydroxystearicacid diglycolamide, N-stearyl ricinoleamide, N-stearyl stearamide and other polyamide waxes, polyethylene oxide waxes, urea urethanes, acrylic resins, amine salts of polymeric polyesters, salts of linear polyaminoamide and polymeric polyester, amide solutions of polycarboxylic acid, alkyl sulfonate, alklyallyl sulfonate, colloidal ester, polyester resin, phenol resin, melamine resin, epoxy resin, urethane resin, styrene butadiene polymers, polyimide resin, and polyester amides.

27. The composition of claim 1 containing from 0.1 to 10 wt % of thixotropic additive.

28. The composition of claim 1 wherein the thixotropic additive consists of organically modified clays.

29. The composition of claim 1 wherein the thixotropic additive consists of Garamite® clays.

30. The composition of claim 1 wherein the thixotropic additive consists of Garamite® 1958.

31. The composition of claim 1 wherein the thixotropic additive consists of silica.

32. The composition of claim 1 wherein the thixotropic additive consists of a mixture of Garamite® clays and silica.

33. The composition of claim 1 wherein a component can be a dispersant.

34. The composition of claim 1 containing from 0.1 to 10 wt % of dispersant.

35. The composition according to claim 1 wherein the pigment or pigments are selected from the group permitted by the US FDA in cosmetics as of Mar. 1, 2010 containing: barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides, D&C Black No. 2, D&C Black No. 3, FD&C Blue No. 1, D&C Blue No. 4, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30. D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, as well as others listed on the FDA color additives website, and Annex IV of the Cosmetic Directive 76/768/EEC, Coloring Agents Permitted in Cosmetics.

35. The composition of claim 1 containing from 0.1 to 10 wt % pigment.

36. The composition of claim 1 containing mixtures of colored pigments and Ti02.

36. The composition in claim 1 with storage times where the pigment does not separate from the homogeneous gel at least 4 times longer at ambient conditions with the thixotropic additive than a composition without the thixotropic additive.

37. The composition in claim 1 with shelf stability where the pigment does not separate from the gel and undergoes syneresis at least 4 times longer at ambient conditions with the thixotropic additive than a composition without the thixotropic additive.

38. The composition in claim 1 with shelf stability where a composition containing the dispersant and the thixotropic additive allows for easier resuspension of the pigment by low shear methods such as shaking and rolling.

39. The composition of claim 1 wherein the gel composition is selected from mono-, di-tri-, and tetra-functional ethylenically unsaturated monomers and oligomers.

40. The composition of claim 1 wherein the gel composition comprises one or more chemicals selected from (meth)acrylic monomers and oligomers.

41. The composition of claim 1 wherein the gel composition comprises a mono-, di-, tri-, or tetra-functional acrylic or methacrylic monomer.

42. The composition of claim 1 wherein the gel composition comprises a polyfunctional polyurethane (meth)acrylate oligomer.

43. The composition of claim 1 wherein the gel composition comprises monomer selected from hydroxyethyl(meth)acrylate, hydroxypropyl(meth)acrylate, trimethylolpropane tri(meth)acrylate, and isobornyl(meth)acrylate.

44. The composition of claim 1 wherein the gel composition comprises aliphatic polyester based urethane diacrylate oligomer.

46. A method of applying the composition from claim 1 onto nails and artificial nails.

47. An artificial colored pigment containing nail coating prepared by curing under actinic radiation a composition of claim 1.
